(19) United States
(12) Patent Application Publication
(10) Pub. No.: US 2004/0121475 A1
(43) Pub. Date: Jun. 24, 2004

(54) Indicator for calcium and magnesium

(76) Inventors: Malcolm Tom Mckechnie, Hull (GB); Gay Joyce Cornelius, Hull (GB); Mark Coke, Hull (GB)

Correspondence Address: Andrew N Parfomak, Norris Mclaughlin & Marcus, 30th Floor, 220 East 42nd Street, New York, NY, 10017, US

(21) Appl. No.: 10/474,157

(22) PCT Filed: Apr. 9, 2002
(86) PCT No.: PCT/GB02/01515

(30) Foreign Application Priority Data
Apr. 10, 2001 (GB) 0108926.7

(51) Int. Cl. G01N 33/20

(52) U.S. Cl. 436/73

(57) ABSTRACT

The invention relates to a product having an anionic metal ion indicator dye bound to it, and use of such products in the detection of metal ions, in particular calcium ions, in a liquid.

The invention relates to a product, preferably a fibre product, having bound to it an anionic metal ion indicator dye, and use of such products in the detection of metal ions, in particular calcium ions, in a liquid.

The ability to test for the presence of metal ions in a liquid is a requirement in many fields, such as water treatment (for example, for detecting heavy metals), laundry (testing for water hardness), health (metal ion concentrations are measured in blood plasma, urine and other bodily fluids). Current tests utilise dyes sensitive to the presence of metal ions such that their colour changes. These may be added directly to a liquid and the colour change detected for a qualitative measurement or the colour change may be titrated, in certain cases, to give a reasonably accurate quantitative measure of the metal ion concentration.

In certain situations it is not desirable to dye the liquid. Alternatively, therefore, dye is impregnated onto strips for dipping into a liquid. However, such strips can not be immersed and left in the liquid since, commonly, metal ion indicator dyes are aqueously soluble and will leach into the liquid.

We have identified a new, quick, simple and effective means for immobilising such dyes.

We present as a feature of the invention a product having bound to it an anionic metal ion indicator dye.

Any number of methods of binding the anionic metal ion indicator dye to the product would be contemplated, including ionic binding.

We present as a preferred feature of the invention a product having a cationic compound to which is ionically bound an anionic metal ion indicator dye.

Preferably the cationic compound is irreversible bound to the product. By “irreversible bound” we mean that less than 20% w/w, 15% w/w, 10% w/w, 5% w/w, preferably less than 1% w/w of the cationic compound is released from the cloth when immersed in an aqueous liquid for 60 minutes, where the temperature is less than 80° C., and the pH is less than <10.5.

The product may be any suitable solid for immersion into a liquid. Suitable solids may be made from a polymer. In a preferred alternative the product is a fibre product, either of a synthetic fibre or a natural fibre.

The fibre product is ideally formed in some way preferably into a sheet, for example a woven, knitted or non-woven sheet. Alternatively it may be twisted into a yarn and, optionally, further formed into a thick yarn, or braid. Alternatively it may be in the form of fibres which may, for example, be tied together. The fibres may be retained in a water-permeable transparent or translucent bag. Most preferably the fibre is a fabric of relatively open form, for example a non-woven fabric or a woven fabric of scrim form.

Where the product is made from a polymer then this may take the form of a sheet, sponge or particulate. It will be appreciated that many polymers may be formed into a fibre. Suitable polymers are those which are positively charged, cationic, and may include polyvinyl pyrolidone and silicas, such as Macrosorb (Ineos Silicas).

Products to which are ionically bound a calcium ion indicator dye are particularly preferred. Calcium ion indicators which can be bound are selected from; metalphthalein, 3,4-dihydroxy-9,10-diazo-2-anthracene sulfonic acid (Alizarin Red S), ammonium purpurate, 1-(1-hydroxy-2-naphthylazo)-5-nitro-2-naphthol-4-sulfonic acid (Eriochrome Black T) and 1-(1-hydroxy-4-methyl-2-phenylazo)-2-napthol-4-sulfonic acid (Calmagite).

A further feature of the invention is a method of detecting the presence of at least one type of metal ion within a liquid which comprises

1) immersing or immersing and removing (preferably just immersing) a product as described herein, and

2) observing the product for any change in colour.

Preferably the liquid is water and in particular it is a wash liquor. Alternatively the water may be; effluent, a bodily liquid or any other liquid in which the presence of a metal ion or its concentration may need to be measured.

It will be appreciated that a colour change may occur when the anionic metal ion indicator dye is bound to the product—this is described in “Dyeing & Chemical Technology of Textile Fibres” Trotman E. R. 1964, Griffin Publishers. The possible colour change seen when a metal ion indicator dye is bound to the product may be different or unexpected, as may the colour change when the product is exposed to the metal ion, compared with the colour change found in solution with the same metal ion indicator dye and metal ion. This in part has to do with the association of the metal ion indicator dye to the product. However, we have found that despite the possibility of such differences a reliable and effective colour change can still successfully occur when the dye is bound to the product.

Preferably the product is able to move freely within the liquid. It could also be in the form of a filter or like body, retained in a fixed orientation, or mounted onto a frame.

In a preferred arrangement the product is are also able to bind metal ions, especially calcium ions so as to soften the water, by also having bound to the cationic compound on the product, not occupied with metal ion indicator dye, metal ion sequestrants. Most preferably the sequestrants are also able to bind further ions, for example magnesium, copper and iron. Preferably any calcium ion sequestrant used is also able to bind such further ions. Alternatively, the product may have two, or more, types of metal ion sequestrant, able to bind different metal ions. In such an arrangement it is preferred that the anionic metal ion indicator dye binds metal ions in preference to any anionic metal ion sequestrant present on the product.

The product may be prepared in any of a number of ways. The cationic compound may be grafted onto the product, for example, cationic compound may be bound to the product by a number of well-known techniques, such as radiation grafting or chemical grafting. Radiation grafting is described in WO 94/12545. Chemical grafting is described in GB 2086954A.

Alternatively, for certain cationic compounds polymeric fibres may be fabricated (for example melt spun) already bearing the cationic compound, as described in EP 486934A. In yet other embodiments the polymeric fibres not bearing the cationic compound may be coated with material which has the cationic compound. The polymeric fibres may, in effect, be regarded as carrying the cationic compound by mechanical adhesion. Alternatively, cationic compound may be attached by cross-linking, as described in EP 992283A.

The anionic metal indicator dye may be bound to the product by impregnating into the products the anionic metal indicator dye, for example by aqueous solution or super critical liquid CO₂.

A suitable technique is described in EP 0210034 for the generation of fibre cloths having bound a cationic compound.

Suitable cloths bearing cationic groups may be purchased easily and anionic metal ion indicator dye added simply by immersing the cloth into a solution of the anionic metal ion indicator dye and rinsing excess dye off. For example “Colour Catcher” ACDO Service Bureau, Bolton B21 8PP, England, www.acdo.co.uk, and “Woolite Colour Catcher” Reckitt Benckiser, Hull, UK, HU8 7DS, www.reckittbenckiser.com.

A further feature of the invention is a process for the preparation of a product, as defined herein, which comprises, immersing a product containing cationic groups into a solution of an anionic metal indicator dye and, optionally, drying the product.

Due to the ionic nature of the binding of the anionic metal indicator dye to the cationic groups we have found that preferably the dye is bound to the cloth at pH conditions of greater than 7, preferably greater than 8, ideally grater than 9.

The concentration of metal ions may be measured by comparing the colour change to a standardised calibration colour change chart.

The metal ion indicator dye may be bound to the product in differing concentrations to provide the possibility of measuring the concentration of metal ions in the liquid directly.

Typical concentrations of metal ion indicator dye which are used when immersing the product to bind the metal ion indicator dye are from 0.1% w/w, preferably 0.03% w/w, to 0.0001% w/w.

The metal ion indicator dye may be bound to the product in simple patterns such as circles or squares or may be deposited in a way so as to create pictures, numbers, letters or words.

### EXPERIMENTAL

### Example 1

Cloths (“Colour Catcher” ACDO Service Bureau, Bolton B21 8PP, England) were immersed in a 0.01% Alizarin Red S solution for 10 minutes, rinsed in 300 ml of deionised water and used wet. A sample of the cloth was immersed in

i) deionised water one at pH 7 and another at pH 10

ii) hard tap water (120 ppm/ml⁻¹?) one at pH 7 and another at pH 10

for 5 minutes. The sample in the deionised water remained purple and the two samples in hard tap water both turned a pink colour.

### Example 2

Cloths (“Colour Catcher” ACDO Service Bureau, Bolton B21 8PP, England) were immersed in a 1.4% Murexide solution for 10 minutes, rinsed in 300 ml of deionised water and used wet and dry. A sample of the cloth was immersed for 5 minutes with gentle agitation in

i) deionised water one at pH 7 and another at pH 10

ii) hard tap water (120 ppm ml⁻¹) one at pH 7 and another at pH 10

for 5 minutes. The cloths in deionised water remained a straw colour whilst cloths in the hard tap water turned pink.

Examples 1 and 2 show a colour response with Ca²⁺ irrespective of pH.

### Example 3

Cloths (“Colour Catcher”) were immersed in 0.1 g Alizarin Red S in 300 ml deionised water, rinsed in 300 ml of deionised water, and dried. The cloth was a red/brown colour.

### Examples 4 & 5

In a similar manner as Example 4, two additional cloths were prepared using Eriochrome Black T, producing a dark blue cloth, and Calmagite, producing a purple/red cloth.

Cloths from Examples 3,4 and 5 were each tested in an electric automatic laundry machine at 60° C. with an “Ariel Essential” tablet on an Easy Care Programme using hard tap water.

Example 3 the cloth after the wash was violet/purple.

Example 4 the cloth after the wash was light purple.

Example 5 the cloth after the wash was violet.

All, therefore, showed a colour change response, even in the presence of laundry detergent, to Ca²⁺ and at an elevated temperature.

### Example 6

0.033 g of Eriochrome Black T was dissolved in 1 l of deionised water. One Woolite Colour Catcher (ReckittBenckiser Hull UK) cloth, dimensions 11.5 cm×21.5 cm, was added to this solution and stirred for 20 minutes. The cloth was removed from the solution and allowed to dry. The cloth treated in the manner above was blue in colour. The cloth remained blue after being immersed in deionised water, but turned purple in water containing calcium. The colour did not bleed significantly from the cloth upon immersion. The purple colour is also evident in the cloth following a washing machine cycle, carried out in hard water, at 60° C. in the presence of a heavy duty laundry detergent, which contains a bleaching compound. Furthermore there is no significant fading of this colour as a consequence of the wash.

When a cloth is soaked in Eriochrome Black T at pH 10 (pH adjusted with NaOH) then greater dye pick-up is seen than without a pH adjustment of the solution. The cloth still becomes blue upon immersion in the dye and changes to purple in hard water as before.

1. A product having cationic groups to which are ionically bound an anionic metal ion indicator dye.

2. A product as claimed in claim 1 wherein the anionic metal indicator dye is ionically bound to a polymer.

3. A product as claimed in claim 1 wherein the anionic metal indicator dye is ionically bound to a fibre, either synthetic or natural.

4. A product as claimed in claim 1 wherein the cationic compound is irreversible bound to the product.

5. A product as claimed in any preceding claim wherein the anionic metal ion indicator dye is sensitive to the presence of calcium ions.

6. A product, as claimed in claim 3 wherein the fibre is formed into a woven, knitted or non-woven sheet.

7. A method of detecting the presence of at least one type of metal ion within a liquid which comprises
1) immersing or inserting and removing (preferably immersing) a product as defined in any claim from 1 to 6, and
2) observing the product for any change in colour.

8. A method as claimed in claim 7 for measuring water hardness by performing the additional step of comparing the colour change to a calibrated colour chart to determine the hardness of the water.

9. A process for the preparation of a product, as defined in any claim from 1 to 6, which comprises, immersing a product containing cationic groups into a solution of an anionic metal indicator dye.

10. A process as claimed in claim 9 wherein the product is dried.

11. A process as claimed in claim 9 or 10, wherein the pH of the solution is adjusted to be greater than 7, preferably greater than 8, ideally greater than 9.

12. A process as claimed in any claim from 9 to 11 wherein the concentration of metal anionic ion indicator dye solution is from 0.1% w/w, preferably 0.03% w/w, to 0.0001% w/w.
